(19) United States
(12) Patent Application Publication
(10) Pub. No.: US 2009/0036905 A1
(43) Pub. Date: Feb. 5, 2009

(54) METHOD OF TISSUE FIXATION USING CINCH STITCHING

(76) Inventor: Reinhold Schmieding, Naples, FL (US)

Correspondence Address: DICKSTEIN SHAPIRO LLP, 1825 EYE STREET NW, Washington, DC, 20006-5403, US

(21) Appl. No.: 12/021,966

(22) Filed: Jan. 29, 2008

Related U.S. Application Data
(60) Provisional application No. 60/898,112, filed on Jan. 30, 2007.

(51) Int. Cl. A61B 17/04

(52) U.S. Cl. 606/144; 606/232

(57) ABSTRACT

A system and method for tissue to bone repair employing cinch stitching combined with at least one knotless fixation device. A suture strand is passed through soft tissue to form a cinch stitch and is then secured in a hole in a bone by the knotless fixation device.

### CROSS REFERENCE TO RELATED APPLICATIONS

This application claims the benefit of U.S. Provisional Application Ser. No. 60/898,112 filed on Jan. 30, 2007, the entire disclosure of which is incorporated by reference in its entirety herein.

### FIELD OF THE INVENTION

The present invention relates to methods of arthroscopic surgery and, more specifically, to an improved method of attaching tissue to bone.

### BACKGROUND OF THE INVENTION

Reattachment of tissue to bone typically requires the surgeon to pass suture material through selected tissue, form a plurality of surgical knots extracorporeally and then move the knots into position adjacent the desired tissue to be sutured. In such procedures, the surgeon must manually tie the knots on the suture strands after the suture is threaded through the selected tissues to be sutured. Knot tying during surgery, particularly arthroscopic surgery, is tedious and time-consuming. There is also a tendency for the knots to deform or collapse as the surgeon manually forces the knots down into the proper position.

Accordingly, a need exists for an improved method for attaching tissue to bone which does not require multiple suture knots and which allows the tendon to be securely in place until the ligaments naturally attach to bone. A method of threading suture through a tendon with maximum suture fixation strength, as well as a method of securing the tissue to bone that allows for accelerated tendon healing to bone are also needed.

### SUMMARY OF THE INVENTION

The present invention provides a system and method for tissue to bone repair employing cinch stitching combined with at least one knotless fixation device. More specifically, the present invention provides methods for tendon to bone fixation by cinch stitching.

Other features and advantages of the present invention will become apparent from the following description of the invention, which refers to the accompanying drawings.

### BRIEF DESCRIPTION OF THE DRAWINGS

FIGS. 1-6 illustrate various steps of a method of arthroscopic repair according to a first exemplary embodiment of the present invention;

FIGS. 7-12 illustrate various steps of a method of arthroscopic repair according to a second exemplary embodiment of the present invention;

FIGS. 13-18 illustrate various steps of a method of arthroscopic repair according to a third exemplary embodiment of the present invention; and

FIGS. 19-24 illustrate various steps of a method of arthroscopic repair according to a fourth exemplary embodiment of the present invention.

### DETAILED DESCRIPTION OF THE PREFERRED EMBODIMENTS

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

The present invention provides a system and method for tissue to bone repair employing cinch stitching combined with at least one knotless fixation device. More specifically, the present invention provides methods for tendon to bone fixation by cinch stitching.

According to an exemplary embodiment, the method comprises the steps of: (i) passing a loop (such as nitinol wire loop) through the labrum; (ii) doubling over a free flexible strand (a suture strand such as FiberWire® suture, a high strength suture sold by Arthrex, Inc. of Naples, Fla., described and claimed in U.S. Pat. No. 6,716,234, the disclosure of which is incorporated by reference) through the nitinol wire loop and shuttling the suture through the tissue; (iii) retrieving the looped end of the doubled-over suture through the cannula; (iv) passing the two suture tails through the suture loop to create a cinch stitch; (v) pulling on the suture tails to slide the cinch knot down to the labrum; and (vi) inserting a knotless fixation device (such as an Arthrex 3.5 mm PushLock™ anchor) into the glenoid to secure the suture and labral tissue to the glenoid.

According to another exemplary embodiment, the method comprises the steps of: (i) passing a loop (such as nitinol wire loop) through the labrum and inserting a doubled over free strand through the nitinol wire loop to form a suture loop; (ii) retrieving the nitinol wire loop to pass the suture loop through the labrum; (iii) pulling the suture loop through the cannula; (iv) inserting a suture retriever instrument through the suture loop to grab both suture tails; (v) passing the suture tails through the suture loop to create a cinch stitch; and (vi) inserting a knotless fixation device into the glenoid to secure the suture and labral tissue to the glenoid.

According to yet an exemplary embodiment, the method comprises the steps of: (i) passing a loop (such as nitinol wire loop) through the labrum; (ii) doubling over a free strand and inserting the resulting loop through the nitinol wire loop, and shuttling the suture through the tissue; (iii) retrieving the looped end of the doubled-over suture through the cannula; (iv) passing the two suture tails through the suture loop to create a cinch stitch; (v) pulling on the suture tails to slide the cinch knot down to the labrum; and (vi) inserting a knotless fixation device into the glenoid to secure the suture and labral tissue to the glenoid.

According to another exemplary embodiment, the method comprises the steps of: (i) inserting a cannula in an anterior portal, doubling over a free strand, loading it into the tip of a device for snaring suture, such as an Arthrex SutureSnare™, and passing it through the labrum; (ii) opening the jaw of the device, releasing the suture loop and pulling the device back out of the labral tissue; (iii) opening the jaws of the device, grabbing the suture loop that was just passed and retrieving the suture loop through the cannula; (iv) passing the two suture tails through the suture loop to create the cinch stitch; (v) pulling on both suture tails to slide the cinch loop down to the labrum; and (vi) inserting the knotless fixation device normally, through the same cannula.

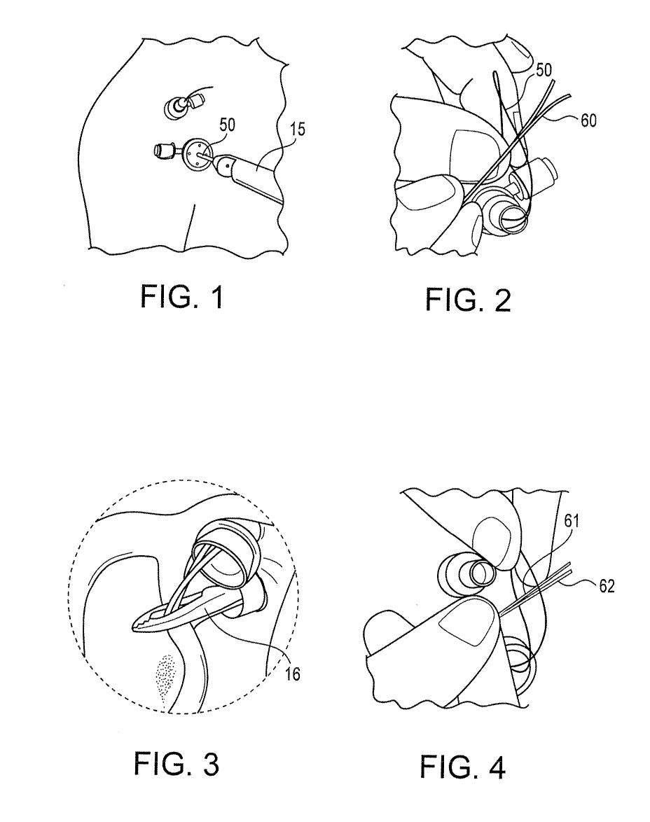

Referring now to the drawings, where like elements are designated by like reference numerals, FIGS. 1-24 illustrate four exemplary methods of attaching a tendon to bone according to the present invention. For exemplary purposes only, the invention will be described below with reference to an arthroscopic glenohumeral joint repair, such as reattachment of labral tissue (cartilage) to the glenoid. However, the invention is not limited to this exemplary embodiment and has applicability to any reattachment of tissue to tissue (such as soft tissue to bone). For exemplary purposes only, the invention will also be described below with reference to particular embodiments employing FiberWire® suture and nitinol. However, the invention is not limited to these exemplary embodiments and has applicability to any flexible strand or combination of strands that are employed in the reattachment of tissue to tissue (such as soft tissue to bone, or soft tissue to soft tissue, for example).

The methods of the present invention distribute suture pressure to tissue, reduce tissue cut, allow for accelerated tissue healing to bone that is achieved with minimal knot tying, and create a better labral bumper when used in conjunction with a knotless fixation device (such as an Arthrex 3.5 mm PushLock™ anchor).

### FIGS. 1-6

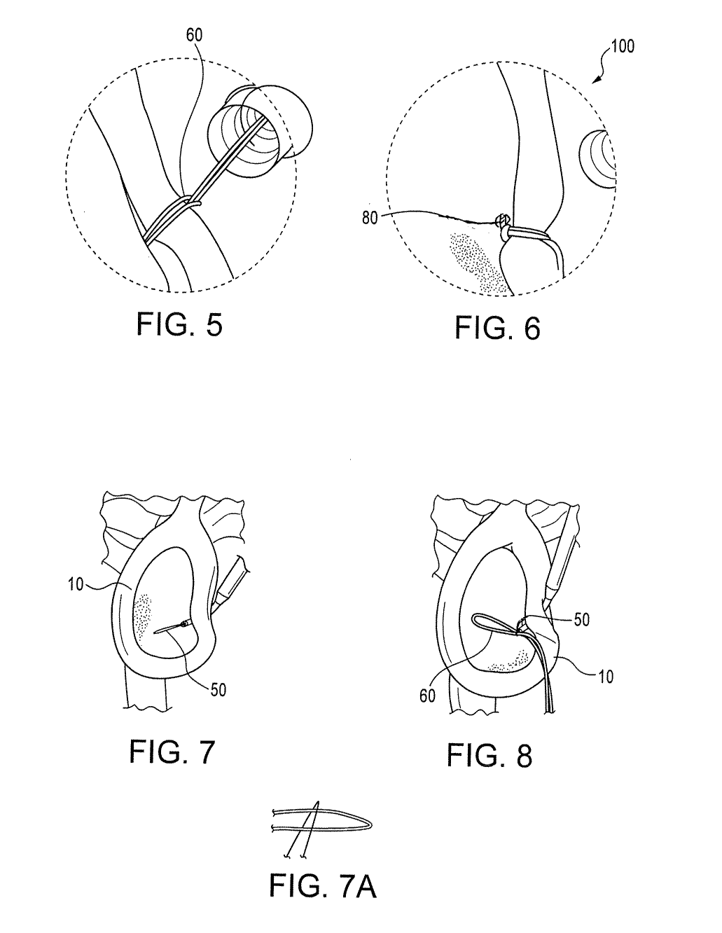

The repair 100 (FIG. 6) according to the first exemplary cinch stitching technique of the present invention includes the following steps:

FIG. 1: A nitinol wire loop 50 is passed through the labrum 10 with a suture passing instrument 15 (such as an Arthrex Suture Lasso™ SD) and the wire 50 is retrieved;
FIG. 2: A free FiberWire® suture 60 is doubled over and the resulting suture tails 61, 62 are inserted through the nitinol wire loop 50 in the anterosuperior cannula. The nitinol wire 50 is pulled back through the anteroinferior cannula to shuttle the suture 60 through the tissue 10;
FIG. 3: A suture retriever instrument 16 (such as an Arthrex KingFisher™ Suture Retriever/Tissue Grasper 16) is used to retrieve the looped end of the doubled-over suture 60 through the anteroinferior cannula;
FIG. 4: The two suture tails 61, 62 are passed through the suture loop 60 to create a cinch stitch 100;
FIG. 5: Both suture tails 61, 62 are pulled to slide the cinch knot down to the labrum 10; and
FIG. 6: A knotless fixation device 80 (such as an Arthrex 3.5 mm PushLock™ anchor 80, as described in U.S. Patent Application Publication No. 2004/0093031, the disclosure of which is incorporated by reference in its entirety) is inserted into the glenoid to secure the suture and labral tissue 10 to the glenoid.

### FIGS. 7-12

The repair 200 (FIG. 12) according to the second exemplary cinch stitching technique (also called an “inside technique”) of the present invention includes the following steps:

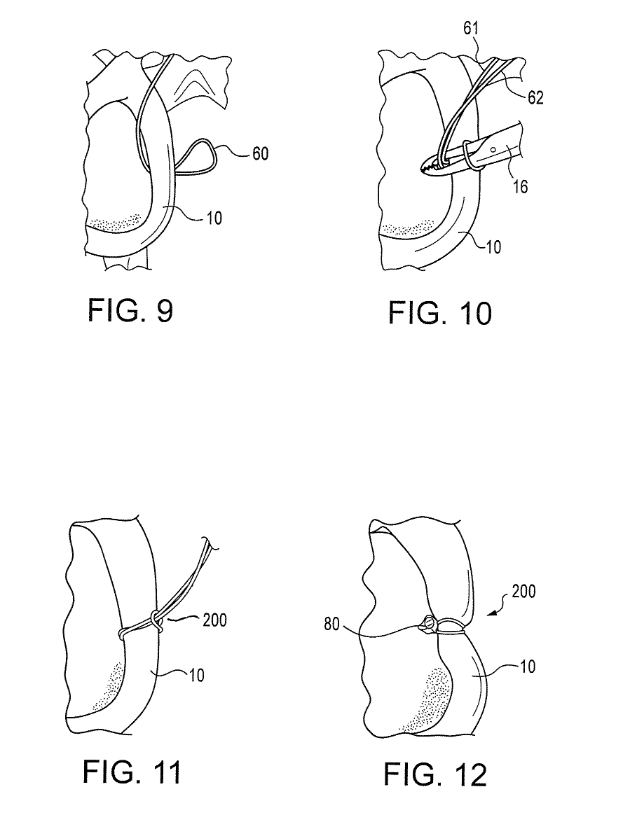

FIG. 7: A nitinol wire loop 50 is passed through the labrum 10 with a suture passing instrument 15 (such as an Arthrex Suture Lasso™ SD 15) and the wire 50 is retrieved; a free FiberWire® suture 60 is doubled over to form a suture loop 60 and the resulting suture loop 60 is inserted through the nitinol wire loop 50;
FIG. 8: The nitinol wire 50 is retrieved to pass the suture loop 60 through the labrum 10;
FIG. 9: The suture loop 60 is pulled back through the cannula and into the joint until it can easily be visualized arthroscopically;
FIG. 10: A suture retriever 16 is inserted through the suture loop 60 and both tails 61, 62 of the suture loop 60 are grabbed;
FIG. 11: The suture tails 61, 62 are retrieved through the suture loop 60 to create the cinch stitch 200; and
FIG. 12: The installation of a knotless fixation device 80 (such as an Arthrex 3.5 mm PushLock™ anchor 80 is completed, as described and claimed in U.S. Patent Application Publication No. 2004/0093031, the disclosure of which is incorporated by reference in its entirety) into the glenoid to secure the suture 60 and labral tissue to the glenoid.

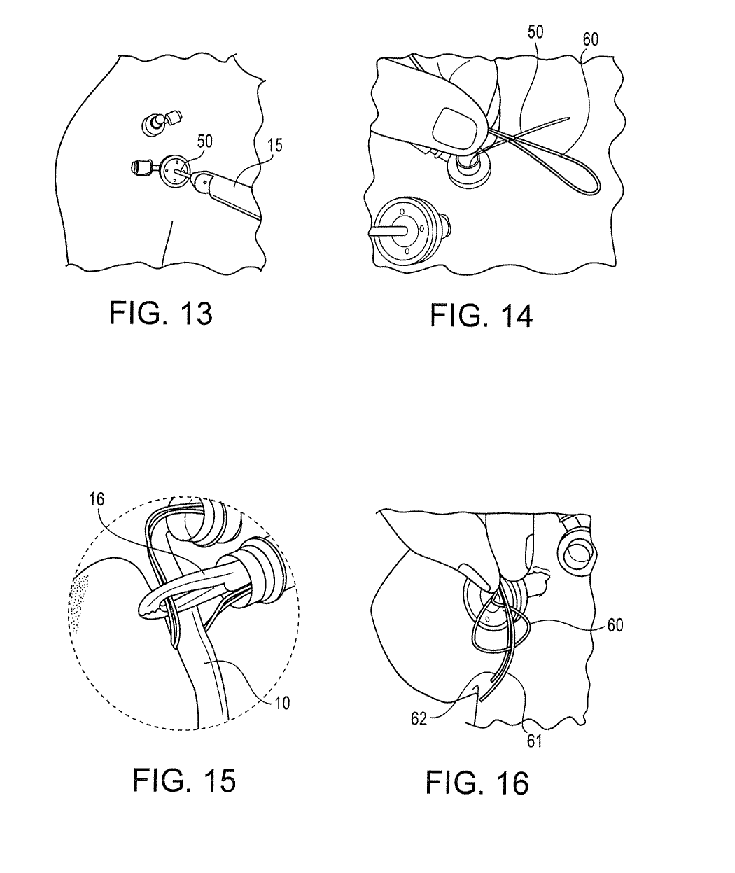

### FIGS. 13-18

The repair 300 (FIG. 18) according to the third exemplary cinch stitching technique of the present invention includes the following steps:

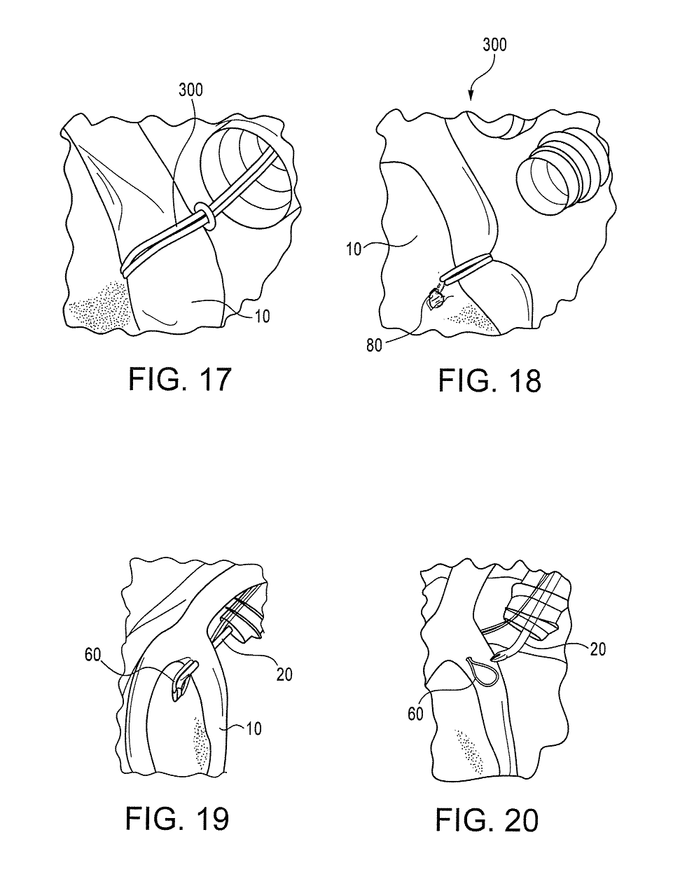

FIG. 13: A nitinol wire loop 50 is passed through the labrum 10 with a suture passing instrument 15 (such as an Arthrex Suture Lasso™ SD 15) and the wire 50 is retrieved;
FIG. 14: A free FiberWire® 60 is doubled over and the resulting FiberWire® loop 60 is inserted through the nitinol wire loop 50 in the anterosuperios cannula; the nitinol wire 50 is pulled back through the anteroinferior cannula to shuttle the suture 60 through the tissue 10;
FIG. 15: A suture retriever instrument 16 (such as an Arthrex KingFisher™ Suture Retriever/Tissue Grasper 16) is used to retrieve the tail ends 61, 62 of the double-over FiberWire 60 through the anteroinferior cannula;
FIG. 16: The two suture tails 61, 62 are passed through the suture loop 60 to create the cinch stitch 300;
FIG. 17: Both suture tails 61, 62 are pulled to slide the cinch knot 300 down to the labrum 10; and
FIG. 18: A knotless fixation device 80 (such as an Arthrex 3.5 mm PushLock™ anchor 80) is inserted into the glenoid to secure the suture and labral tissue 10 to the glenoid.

### FIGS. 19-24

The repair 400 (FIG. 24) according to the fourth exemplary cinch stitching technique (also called a “single portal technique”) of the present invention employs an Arthrex SutureSnare™ (or BirdBeak™) which is used to easily complete a cinch stitch through a single portal. The knotless fixation device 80 (e.g., a PushLock™ anchor 80) can then be installed through the same portal. This technique includes the following steps:

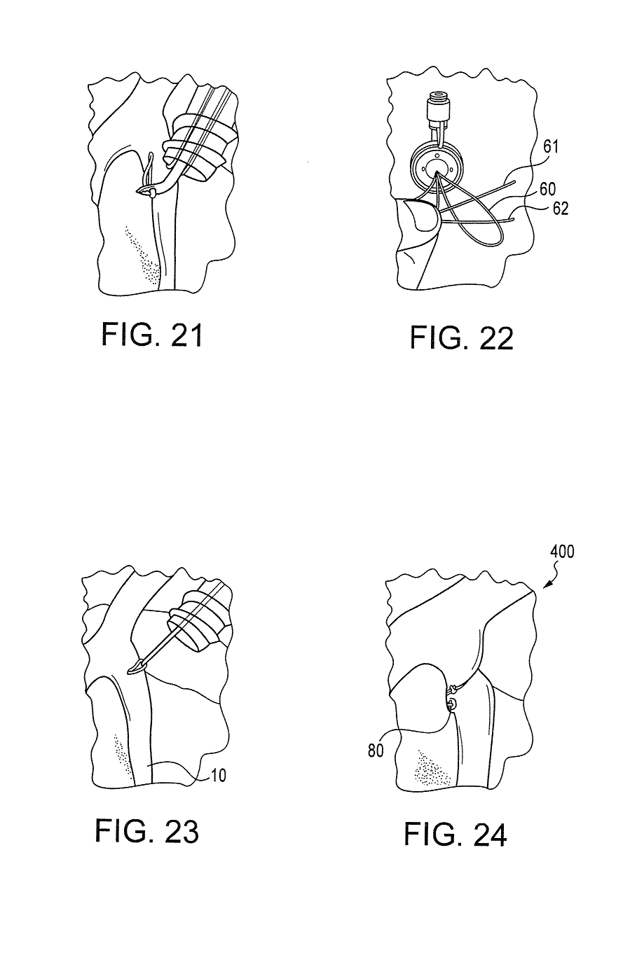

FIG. 19: A 8.25 mm cannula is inserted in an anterior portal; a free FiberWire® 60 is doubled over and loaded into the tip of a 60° SutureSnare™ 20 and passed through the labrum 10;
FIG. 20: The button on the rear of the SutureSnare™ 20 is pressed to open the jaw and release the suture loop 60; the SutureSnare™ 20 is pulled back out of the labral tissue 10;
FIG. 21: The jaws of the SutureSnare™ 20 are opened and the suture loop 60 that was just passed is grabbed; the knob on the handle of the SutureSnare™ handle is turned to lock the jaws; the suture loop 60 is retrieved through the cannula;
FIG. 22: The two suture tails 61, 62 are pulled through the suture loop 60 to create the cinch stitch 400;
FIG. 23: Both suture tails 61, 62 are pulled to slide the cinch loop 400 down to the labrum 10; and
FIG. 24: a knotless fixation device 80 (such as an Arthrex 3.5 mm PushLock™ anchor 80) is inserted through the same cannula and into the glenoid, to secure the suture 60 and labral tissue 10 to the glenoid.

As mentioned previously, the strand or suture 60 employed in the method of the present invention may be formed of any flexible material. In the preferred embodiment, the sutures are formed of a high strength suture material such as FiberWire® suture, sold by Arthrex, Inc. of Naples, Fla., and described in U.S. Pat. No. 6,716,234, the disclosure of which is incorporated by reference herein. The high strength suture may be available in various lengths and, preferably, is a #2 FiberWire® suture strand. FiberWire® suture is formed of an advanced, high-strength fiber material, namely ultrahigh molecular weight polyethylene (UHMWPE), sold under the tradenames Spectra (Honeywell) and Dyneema (DSM), braided with at least one other fiber, natural or synthetic, to form lengths of suture material. The preferred FiberWire® suture includes a core within a hollow braided construct, the core being a twisted yarn of UHMWPE. The suture may optionally include filaments of various colors.

Preferably, the fixation device 80 employed in the embodiments described above may be a knotless fixation device (such as an Arthrex 3.5 mm PushLock™ anchor, or Arthrex SwiveLock™ anchor), or a combination of the PushLock™ and the SwiveLock™ anchors, or a combination of a PushLock™ and SwiveLock™ anchor with another other fixation device, such as Arthrex 5.5 mm Bio-Corkscrew® FT anchor, for example. The Arthrex PushLock™ anchor is disclosed and described in U.S. Patent Application Publication No. 2004/0093031, the disclosure of which is hereby incorporated by reference in its entirety, and the Arthrex SwiveLock™ anchor is disclosed and described in U.S. Application Publication No. 2007/0191849, the disclosure of which is also hereby incorporated by reference in its entirety. The result is a quick, secure and low profile repair with excellent contact between tendon and bone.

In exemplary embodiments, the fixation device 80 may be employed with additional fixation devices such as a fully-threaded bioabsorbable suture anchor having a loop inserted into the suture anchor, as disclosed and described in U.S. Patent Application Publication No. 2007/0060922, the disclosure of which is hereby incorporated by reference in its entirety. In other embodiments, the fixation device 80 may employed with an Arthrex Biocorkscrew™, described and claimed in U.S. Patent Application Publication No. 2004/0106950, the disclosure of which is hereby incorporated by reference in its entirety, having an eyelet and loaded with a single or double strands of sutures.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Accordingly, it is not intended that the present invention be limited to the illustrated embodiments, but only by the appended claims.

What is claimed as new and desired to be protected by Letters Patent of the United States is:

1. A knotless method of attaching soft tissue to bone, comprising:
providing a flexible strand;
forming at least one cinch stitch through soft tissue with the flexible strand; and
securing at least a portion of the cinch stitch in a hole in bone by a knotless fixation device.

2. The method of claim 1, wherein the knotless fixation device is an Arthrex PushLock™ anchor or a SwiveLock™ anchor.

3. The method of claim 1, wherein the flexible strand is a high strength suture.

4. The method of claim 1, wherein the soft tissue is labral tissue and the bone is the glenoid.

5. A method of attaching soft tissue to bone, comprising:
passing a first flexible loop through soft tissue;
passing a second flexible loop through the soft tissue by employing the first flexible loop;
passing free ends of the second flexible loop through the second flexible loop and over a portion of the soft tissue; and
securing at least a part of the second flexible loop in a hole in bone by a fixation device.

6. The method of claim 5, wherein the first flexible loop comprises nitinol.

7. The method of claim 5, wherein the second flexible loop is comprised of suture.

8. The method of claim 5, wherein the second flexible loop is a high strength suture comprising ultrahigh molecular weight polyethylene.

9. The method of claim 5 further comprising the step of pulling on at least a free end of the second flexible loop to shorten a length of the second flexible loop and to form a cinch stitch.

10. The method of claim 5, wherein the step of securing at least a part of the second flexible loop in a hole in bone is performed without tying any knots.

11. The method of claim 5, wherein the least a part of the second flexible loop is fixedly secured to the fixation device prior to insertion of the fixation device into the bone.

12. The method of claim 5, wherein the fixation device is a knotless fixation device.

13. The method of claim 5, wherein the fixation device is a press-in suture anchor.

14. The method of claim 5, wherein the fixation device is a suture anchor with a swivel implant.

15. A knotless method of attaching soft tissue to bone, comprising:
passing a first flexible loop through soft tissue;
providing a flexible strand in the vicinity of soft tissue and doubling over the flexible strand to form a double-over strand with free ends;
inserting the free ends of the double-over strand through the first flexible loop;
subsequently passing the free ends and the double-over strand through the soft tissue by employing the first flexible loop;
sliding free ends of the double-over strand through the double-over strand and over a lateral portion of the soft tissue, to form a cinch stitch;
pulling on both free ends to decrease a distance between the cinch stitch and the soft tissue; and
securing the free ends in a hole in bone by at least one knotless fixation device.

16. The method of claim 15, wherein the first flexible loop comprises nitinol.

17. The method of claim 15, wherein the flexible strand is comprised of suture.

18. The method of claim 15, wherein the flexible strand is a high strength suture comprising ultrahigh molecular weight polyethylene.

19. The method of claim 15, wherein the knotless fixation device is a PushLock™ anchor or a SwiveLock™ anchor.

20. A knotless method of attaching soft tissue to bone through a single arthroscopic portal, comprising:
doubling over a flexible strand to form a double-over strand with two free ends;
passing the double-over strand with two free ends through soft tissue;
sliding the two free ends through the double-over strand and over a portion of the soft tissue, to form a cinch stitch; and
securing at least a part of the double-over strand in a hole in bone by a knotless fixation device.

21. The method of claim 20, wherein the knotless fixation device is a PushLock™ anchor or a SwiveLock™ anchor.

22. The method of claim 20, wherein the flexible strand is a high strength suture comprising ultrahigh molecular weight polyethylene.

23. The method of claim 20 further comprising the step of pulling on the free ends to decrease the distance between the cinch stitch and the soft tissue.

24. The method of claim 20, wherein the soft tissue is labral tissue and the bone is the glenoid.

25. An apparatus for attachment of soft tissue to bone, comprising:
a suture strand;
a suture passing device configured to pass a loop of the suture strand through soft tissue to form a cinch stitch; and
a knotless fixation device for securing at least a portion of the suture strand in a hole in a bone.

26. The apparatus of claim 25, wherein the knotless fixation device is a PushLock™ anchor or a SwiveLock™ anchor.

27. The apparatus of claim 25, wherein the suture strand is a high strength suture comprising ultrahigh molecular weight polyethylene.

28. The apparatus of claim 25, wherein the suture passing device is a nitinol wire loop.

29. The apparatus of claim 25, wherein the suture passing device is a suture passing instrument.
